(19) United States
(12) Patent Application Publication
(10) Pub. No.: US 2014/0147882 A1
(43) Pub. Date: May 29, 2014

(54) METHOD AND DEVICE FOR DETECTION AND QUANTIFICATION OF THERMODURIC MICROORGANISMS IN A PRODUCT

(71) Applicants: James Nial Hynes, Ovens, County Cork (IE); Dmitri Boris Papkovsky, Blarney County Cork (IE)

(72) Inventors: James Nial Hynes, Ovens, County Cork (IE); Dmitri Boris Papkovsky, Blarney County Cork (IE)

(73) Assignee: Luxcel Biosciences Ltd., Cork (IE)

(21) Appl. No.: 14/233,602

(22) PCT Filed: Jul. 18, 2011
(86) PCT No.: PCT/EP2011/062233

§ 371 Date: Jan. 17, 2014

(51) Int. Cl. C12Q 1/04

(52) U.S. Cl. CPC C12Q 1/04
USPC 435/34

(57) ABSTRACT

Method of detecting the presence of thermoduric microorganisms in a product that includes the steps of (i) placing an aliquot A of a product into a vessel 10 equipped with a probe 30 sensitive to a thermoduric microorganism metabolite, (ii) pasteurizing the aliquot A within the vessel 10, (iii) incubating the pasteurized aliquot A within the vessel 10 for an incubation period, and (iv) periodically interrogating the probe 30 during the incubation period.

### BACKGROUND

A number of food products cannot be sterilized without adversely affecting the quality and/or taste of the product. Such foods, such as milk, are often pasteurized in order to reduce the number of viable pathogens and slow microbial growth without adversely affecting the taste and quality of the product.

There are various pasteurization techniques in use today. These include Ultra High Temperature (UHT), High Temperature/Short Time (HTST), Vat or Batch (LTLT), and Extended Shelf Life (ESL). The UHT pasteurization technique heats the product to 135° C. (275° F.) for a minimum of one second. The HTST pasteurization technique heats the product to 72° C. (161° F.) for 15 to 20 seconds. The Vat or Batch pasteurization technique heats the product to 63° C. (146° F.) for 30 minutes. The ESL pasteurization technique uses even lower temperatures but in combination with microbial filtration.

While pasteurization is effective for enhancing shelf-life and reducing microbial risks, some bacteria survive pasteurization. Such bacteria are known as thermoduric bacteria and are most commonly associated with some contamination source. The standard test for detecting and enumerating thermoduric bacteria is the Laboratory Pasteurization Count (LPC), which serves as an indicator of the effectiveness of farm sanitation and hygiene procedures. Under laboratory conditions pasteurization at lower temperatures is usually easier to implement. However, the use of lower pasteurization temperatures can be associated with a higher risk of residual contamination and hence can require more strict control measures.

LPC typically involves heating a rack of approximately 5 ml samples, each retained within in a sampling bottle, to 63° C. in a water bath for 30 min, followed by immediate cooling. An aliquot of each individual temperature treated sample is then withdrawn from the bottle and deposited into an agar plate, followed by prolonged incubation (typically 24 to 72 hrs) and subsequent colony counting.

While generally effective for detecting and enumerating thermoduric bacteria, LPC is relatively slow and labor-intensive as it includes numerous steps involving manipulation of the samples, and results in a subjective readout.

Accordingly, a need exists for an improved method of detecting and enumerating thermoduric bacteria.

### SUMMARY OF THE INVENTION

The invention is directed to the detection of thermoduric microorganisms in a product with minimal manipulation of the sample. A first embodiment of the invention is a method of detecting the presence of thermoduric microorganisms in a product. The method includes the steps of (i) placing an aliquot of the product into a vessel equipped with an optical probe sensitive to a thermoduric microorganism metabolite, (ii) pasteurizing the aliquot within the vessel, (iii) incubating the pasteurized aliquot within the vessel for an incubation period, and (iv) periodically interrogating the probe during the incubation period. The interrogations measure changes in the probe reflective of changes in concentration of a thermoduric microorganism metabolite within the aliquot, thereby indicating the presence of viable thermoduric microorganisms in the aliquot.

Thermoduric microorganisms in the product prior to incubation can be enumerated by converting measured changes in the probe to a concentration of thermoduric microorganisms in the pasteurized aliquot based upon a known conversion algorithm.

Interrogation of the probe is preferably effected remotely, through the walls of the vessel, in order to eliminate the need to physically contact the aliquot during the testing period.

A second embodiment of the invention is a method for comparatively detecting the presence of thermoduric microorganisms and total microorganisms in a product. The method includes the steps of (i) obtaining a sample of the product, (ii) placing a first aliquot of the sample into a first retention chamber equipped with a first probe sensitive to a thermoduric microorganism metabolite, (iii) placing a second aliquot of the sample into a second retention chamber equipped with a second probe sensitive to a target-analyte, (d) pasteurizing the first aliquot within the first retention chamber but not the second aliquot, (e) incubating the pasteurized first aliquot within the first retention chamber and the second aliquot within the second retention chamber for an incubation period, and (f) periodically interrogating both probes during the incubation period, wherein the interrogations measure changes in the probe reflective of changes in concentration of a thermoduric microorganism metabolite within the first aliquot and changes in concentration of a target-analyte within the second aliquot, with such changes in concentration indicative of the presence of viable thermoduric microorganisms in the first aliquot and the presence of total viable microorganisms in the second aliquot.

Thermoduric microorganisms and total microorganisms in the product prior to incubation can be enumerated by converting measured changes in the first probe to a concentration of thermoduric microorganisms in the pasteurized first aliquot based upon a known conversion algorithm, and converting measured changes in the second probe to a concentration of total microorganisms in the second aliquot prior to incubation based upon a known conversion algorithm.

Interrogation of the probe is preferably effected remotely, through the walls of the vessel, in order to eliminate the need to physically contact the aliquot during the testing period.

### BRIEF DESCRIPTION OF THE DRAWINGS

FIG. 1 is a side view of one embodiment of a tool in accordance with this invention.

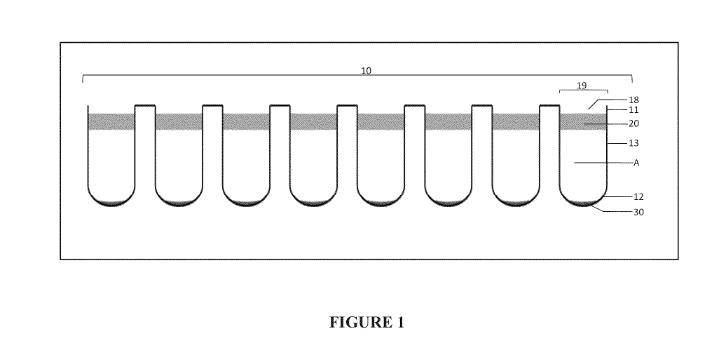

FIG. 2 is a generic graph of temperature over time for an exemplary process for detecting thermoduric microorganisms in a product in accordance with this invention. Phase 1 (t₀ to t₁) indicates sample preparation steps (s) (no temperature control and no probe interrogation). Phase 2 (t₁ to t₂) indicates pasteurisation step (temperature ramp for defined time period without probe interrogation). Phase 3 (t₂ to t₃) indicates incubation period (incubation temperature with periodic probe interrogation).

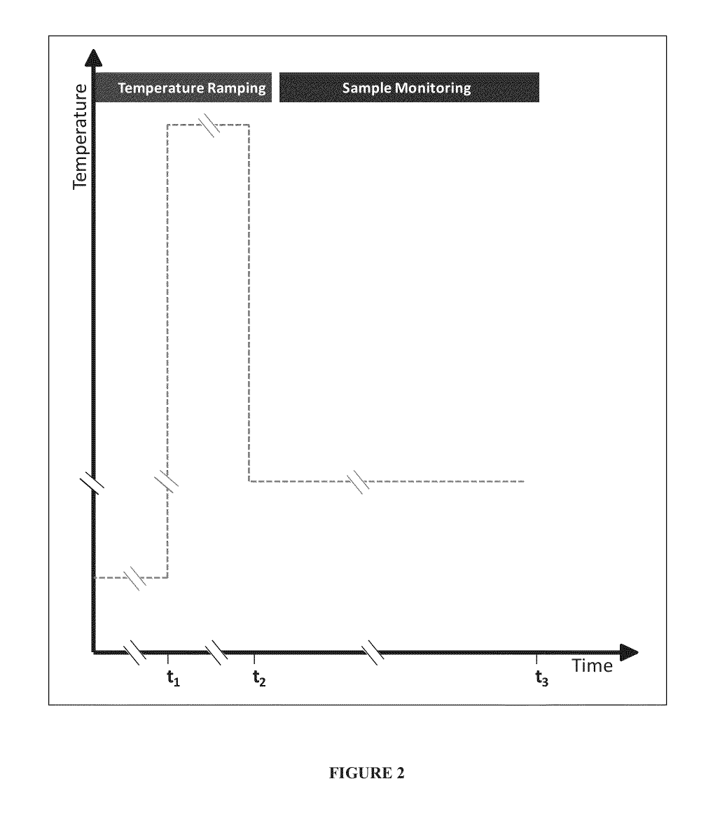

FIG. 3 is a generic graph of probe signal over time for an exemplary detection of thermoduric microorganisms during Phase 3 (t₂ to t₃) in a product in accordance with this invention. Exemplary signal profiles for both a positive aliquot (i.e., an aliquot with sufficient thermoduric microorganisms to grow after pasteurization and consume substantially all target-analyte within the aliquot during the incubation period (Phase 3 (t₂ to t₃))), and a negative aliquot (i.e., an aliquot with insufficient thermoduric microorganisms to grow after pasteurization and consume substantially all target-analyte within the aliquot during the incubation period (Phase 3 (t₂ to t₃))).

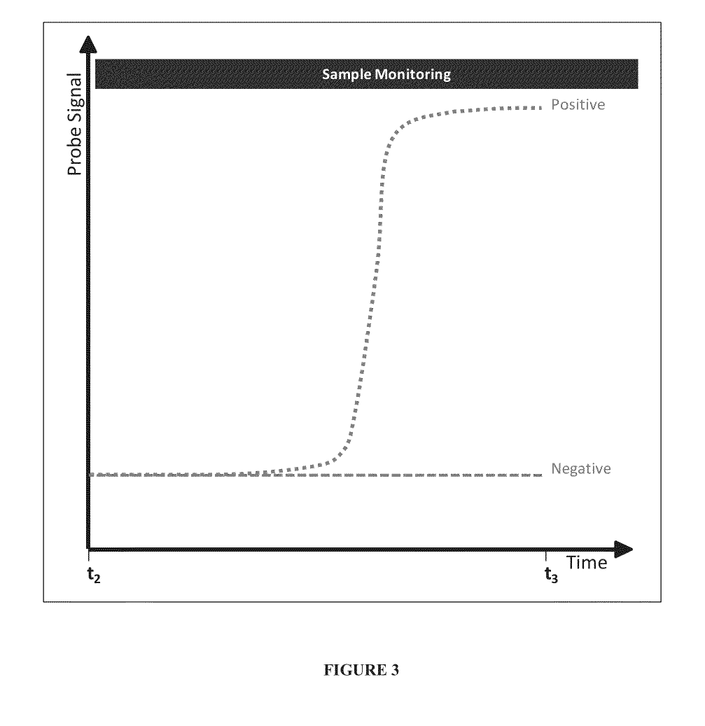

### DETAILED DESCRIPTION OF A PREFERRED EMBODIMENT

### Definitions

As used herein, including the claims, the term “pasteurization” means heating a product, typically a liquid food product susceptible to degradation at sterilization temperatures, to a specific elevated temperature below that required for sterilization for a defined length of time to slow microbial growth.

As used herein, including the claims, the phrase “Vat Pasteurization” (VatP) or “Batch Pasteurization” (LTLT) means heated to and held at 63° C. (145° F.) for 30 minutes.

As used herein, including the claims, the phrase “High Temperature Short Time Pasteurization” (HTST) means heated to and held at 72° C. (162° F.) for 15 seconds.

As used herein, including the claims, the phrase “Ultra Pasteurization” (UP) means heated to and held at 138° C. (280° F.) for 2 seconds.

As used herein, including the claims, the phrase “thermoduric microorganism metabolite” means a molecule consumed or produced during thermoduric microorganism metabolism.

As utilized herein, including the claims, the phrase “surface-to-volume ratio” means the amount of surface per unit volume.

As utilized herein, including the claims, the phrase “elevated surface-to-volume ratio” means a surface-to-volume ratio of greater than 12:1.

As used herein, including the claims, the term “target-analyte” refers to a chemical substance, typically O₂, CO₂ or H⁺, constituting a thermoduric microorganism metabolite and capable of modulating the optical signal emanating from an optically-active material such as a photoluminescentprobe. The modulating effect may be achieved by quenching, enhancement, (de)protonation or other means.

### Nomenclature

10 Vessel
11 Top of Vessel
12 Bottom of Vessel
13 Diathermal Sidewalls of Vessel
18 Opening into Retention Chamber
19 Retention Chamber
19₁ First Retention Chamber
19₂ Second Retention Chamber
20 Cap
30 Probe
30₁ First Probe
30₂ Second Probe
A Aliquot of Product
A₁ First Aliquot of Product
A₂ Second Aliquot of Product

### Description

Tool

The invention employs a tool specially adapted for use in detecting the presence of microorganisms, particularly thermoduric microorganisms, in a product. The tool is a small vessel 10 defining a retention chamber 19 and equipped with a probe 30 sensitive to a target-analyte in operable communication with the retention chamber 19. The top 11 of the vessel 10 is open for allowing access to the retention chamber 19. A cap 20 or other sealing device may be provided for sealing the top 11 of the retention chamber 19.

Suitable vessels 10 include vials, cuvettes, multi-well plates (e.g., 6, 12, 24, 48, 96 and 384 well plates), and the like.

The vessel 10 is constructed, configured and arranged to withstand pasteurization temperatures and temperature profiles, and permit any contents placed within the retention chamber 19 of the vessel 10 to be quickly heated and cooled through the diathermal sidewall(s) 13 of the vessel 10. The vessel 10 is preferably constructed, configured and arranged such that thermal equilibration of an aliquot A placed into the retention chamber 19 of the vessel 10 can be achieved within ½ of the pasteurization time period, preferably within ¼^{th} of the pasteurization time period and most preferably even quicker than this.

The retention chamber 19 is preferably sized and configured to facilitate quick heating and cooling of any contents by providing an elevated surface-to-volume ratio, most preferably a high surface-to-volume ratio.

The probe 30 can be any device capable of sensing and reporting changes in a target-analyte concentration within an enclosed volume. In a preferred embodiment, the probe 30 is an optically-active, target-analyte sensitive material configured and arranged to experience changes in target-analyte concentration or partial pressure P_{A} in an aliquot A placed within the retention chamber 19 of the vessel 10. The analyte-sensitive material is preferably a photoluminescent dye embedded within an analyte permeable polymer matrix. Since the preferred type of probe 30 is an optically-active, target-analyte sensitive material, and the most frequent target-analyte of interest is oxygen, the balance of the disclosure shall be based upon a photoluminescent oxygen quenched probe 30 without intending to be limited thereby.

The oxygen-sensitive photoluminescent dye may be selected from any of the well-known oxygen sensitive photoluminescent dyes. One of routine skill in the art is capable of selecting a suitable dye based upon the intended use of the probe 30. A nonexhaustive list of suitable oxygen sensitive photoluminescent dyes includes specifically, but not exclusively, ruthenium(II)-bipyridyl and ruthenium(II)-diphenylphenanothroline complexes, porphyrin-ketones such as platinum(II)-octaethylporphine-ketone, platinum(II)-porphyrin such as platinum(II)-tetrakis(pentafluorophenyl)porphine, palladium(II)-porphyrin such as palladium(II)-tetrakis(pentafluorophenyl)porphine, phosphorescent metallocomplexes of tetrabenzoporphyrins, chlorins, azaporphyrins, and long-decay luminescent complexes of iridium(III) or osmium(II).

Typically, the hydrophobic oxygen-sensitive photoluminescent dye is compounded with a suitable oxygen-permeable and hydrophobic carrier matrix. The carrier matrix, indeed the entire probe 30, must be able to withstand the pasteurisation and incubation conditions. Again, one of routine skill in the art is capable of selecting a suitable oxygen-permeable hydrophobic carrier matrix based upon the intended use of the probe 30 and the selected dye. A nonexhaustive list of suitable polymers for use as the oxygen-permeable hydrophobic carrier matrix includes specifically, but not exclusively, polystryrene, polycarbonate, polysulfone, polyvinyl chloride and some co-polymers.

When the probe 30 is based on the quenching of photoluminescence by an analyte, the vessel 10, or at least that portion of the vessel 10 coated with the probe 30, must allow radiation at the excitation and emission wavelengths to be transmitted to and received from the probe 30 with minimal interference. The probe 30 is preferably positioned within the retention chamber 19 proximate the bottom end 12 of the vessel 19.

The vessel 10 may be formed from a wide variety of materials, such as various plastics (e.g., polypropylene or polyethylene terphthalate), glass, etc.

The radiation emitted by an excited probe 30 can be measured in terms of intensity and/or lifetime (rate of decay, phase shift or anisotropy), with measurement of lifetime generally preferred as a more accurate and reliable measurement technique.

Instruments (not shown) for interrogating probes 30 based on the quenching of photoluminescence by an analyte are well known and commercially available from various sources, including bioMérieux SA of France and Mocon, Inc. of Minneapolis, Minn.

### Method

In a first embodiment, the invention is a method of detecting the presence of thermoduric microorganisms in a product. The method includes the steps of (i) placing an aliquot A of the product into a vessel 10 equipped with a probe 30 sensitive to a target-analyte, (ii) pasteurizing the aliquot A within the vessel 10, (iii) incubating the pasteurized aliquot A within the vessel 10 for an incubation period, typically at temperatures of between 30° to 55° C., and (iv) periodically interrogating the probe 30 during the incubation period.

The aliquot A within the vessel 10 may be pasteurized by any of the generally accepted pasteurization techniques, including Ultra High Temperature (UHT), High Temperature/Short Time (HTST), and Vat or Batch. For compliance purposes, it is preferred to use the same time and temperature values utilized with the currently employed LPC (i.e., 63° C. for 30 min). In order to avoid the problems associated with use of a water bath to achieve pasteurization temperatures, it is preferred to use a dry block heater to achieve and maintain stable pasteurization and incubation temperatures.

The interrogations measure changes in the probe 30 reflective of changes in concentration of a target-analyte within the aliquot A, thereby indicating the presence of viable thermoduric microorganisms in the aliquot A. Conversion algorithms used to convert the measured probe emissions to an oxygen concentration are well know to and readily developable by those with routine skill in the art.

If desired, the concentration of thermoduric microorganisms in the product prior to incubation can be determined by analyzing measured changes in the probe 30 in communication with the pasteurized aliquot A. Conversion algorithms for converting the measured emissions to a concentration of microorganisms in a sample are well know to and readily developable by those with routine skill in the art.

Nutrients can be added to the aliquot A prior to pasteurization for purposes of promoting growth of thermoduric microorganisms (or subsets thereof) in the aliquot A. Such nutrients, commonly referenced as culture media, are widely available from a number of sources. Persons of routine skill in the art would be able to identify and select suitable nutrients for incorporation into the aliquot A.

It is generally preferred to hermetically seal the aliquot A within the retention chamber 19 to facilitate handling (e.g., avoid spillage), prevent evaporation, and prevent both bacterial and target-analyte contamination of the aliquot A. The open top 11 of the retention chamber 19 can be sealed by any of the well know sealing techniques capable of withstanding the thermal treatment, including specifically but not exclusively a screw cap 20, a heat or adhesive sealing foil (not shown), layer of mineral oil (not shown), etc.

The method is particularly suited for use in safety testing of liquid food products intended for human consumption, such as milk, juices and beverages.

In a second embodiment, the invention is a method for comparatively detecting the presence of thermoduric microorganisms and total microorganisms in a product. The method includes the steps of (i) obtaining a sample of the product, (ii) placing a first aliquot A₁ of the sample into a first retention chamber 19₁ equipped with a first probe 30₁ sensitive to a target-analyte, (iii) placing a second aliquot A₂ of the sample into a second retention chamber 19₂ equipped with a second probe 30₂ sensitive to a target-analyte, (d) pasteurizing the first aliquot A₁ within the first retention chamber 19₁ but not the second aliquot A₂, (e) incubating the pasteurized first aliquot A₁ within the first retention chamber 19₁ and the second aliquot A₂ within the second retention chamber 19₂ for an incubation period, and (f) periodically interrogating both probes 30₁ and 30₂ during the incubation period, wherein the interrogations measure changes in the probe 30₁ or 30₂ reflective of changes in concentration of a target-analyte within the aliquot A₁ or A₂ respectively, with such changes in concentration indicative of the presence of viable thermoduric microorganisms in the first aliquot A₁ and the presence of total viable microorganisms in the second aliquot A₂.

As with the first embodiment, the first aliquot A₁ may be pasteurized by any of the generally accepted pasteurization techniques, including Ultra High Temperature (UHT), High Temperature/Short Time (HTST), and Vat or Batch. For compliance purposes, it is preferred to use the same time and temperature values utilized with the currently employed LPC (i.e., 63° C. for 30 min). In order to avoid the problems associated with use of a water bath to achieve pasteurization temperatures, it is preferred to use a dry block heater to achieve and maintain pasteurization temperatures.

The interrogations measure changes in the probe 30 reflective of changes in concentration of a target-analyte within the aliquot A, thereby indicating the presence of viable thermoduric microorganisms in the first aliquot A₁ and the presence of total microorganisms in the second aliquot A₂. Again, conversion algorithms used to convert the measured probe emissions to an oxygen concentration are well know to and readily developable by those with routine skill in the art.

If desired, the concentration of thermoduric microorganisms in the product prior to incubation can be determined by analyzing measured changes in the first probe 30₁ to a concentration of thermoduric microorganisms in the pasteurized first aliquot A₁, and the concentration of total microorganisms in the product prior to incubation can be determined by analyzing measured changes in the second probe 30₂ to a concentration of total microorganisms in the pasteurized second aliquot A₂. Conversion algorithms used to convert the measured emissions to a concentration of microorganisms (thermoduric or total) in a sample are well know to and readily developable by those with routine skill in the art.

Nutrients can be added to both aliquots A₁ and A₂ prior to pasteurization/incubation for purposes of promoting growth of relevant microorganisms in the product.

As with the first embodiment, it is generally preferred to hermetically seal the aliquots A₁ and A₂ within the corresponding retention chamber 19₁ and 19₂ to prevent both bacterial and target-analyte contamination of the aliquot A₁ and A₂.

As with the first embodiment, the method is particularly suited for use in safety testing of food products intended for human consumption, such as milk.

We claim:

1. A method of detecting the presence of thermoduric microorganisms in a product, comprising the steps of:
(a) placing an aliquot of the product into a vessel equipped with an optical probe sensitive to a thermoduric microorganism metabolite,
(b) pasteurizing the aliquot within the vessel,
(c) incubating the pasteurized aliquot within the vessel for an incubation period, and
(d) periodically interrogating the probe during the incubation period, wherein the interrogations measure changes in the probe reflective of changes in concentration of a thermoduric microorganism metabolite within the aliquot, with such changes in concentration indicative of the presence of viable thermoduric microorganisms in the aliquot.

2. The method of claim 1 wherein the thermoduric microorganism metabolite is oxygen.

3. The method of claim 1 further comprising the steps of (i) converting measured changes in the probe to a concentration of thermoduric microorganisms in the pasteurized aliquot prior to incubation based upon a known conversion algorithm, and (ii) reporting the ascertained concentration of thermoduric microorganisms.

4. The method of claim 1 further comprising the step of hermetically sealing the aliquot within the retention chamber of the vessel prior to pasteurization.

5. The method of claim 1 wherein the product is a food product intended for animal or human consumption.

6. The method of claim 5 wherein the product is milk.

7. The method of claim 1 wherein the vessel is a vial, cuvette or multi-well plate.

8. The method of claim 1 wherein the vessel defines a retention chamber having an elevated surface-to-volume ratio.

9. The method of claim 1 wherein pasteurization is effected at a defined temperature for a defined time period and thermal equilibration of the aliquot in the vessel is achieved within ½ of the pasteurization time period.

10. The method of claim 1 wherein pasteurization is effected at a defined temperature for a defined time period and thermal equilibration of the aliquot in the vessel is achieved within ¼^{th} of the pasteurization time period.

11. The method of claim 7 wherein the vessel is a multi-well plate having more than 40 wells.

12. The method of claim 1 wherein (i) the vessel defines a retention chamber having an open top end and a closed bottom end, and (ii) the probe is positioned within the retention chamber proximate the bottom end.

13. The method of claim 2 wherein the probe is an oxygen sensitive photoluminescent dye.

14. The method of claim 13 wherein the oxygen sensitive photoluminescent dye is an oxygen sensitive transition metal complex.

15. The method of claim 14 wherein the oxygen sensitive transition metal complex is selected from the group consisting of a ruthenium bipyridyl, a ruthenium diphenylphenanotroline, a platinum porphyrin, a palladium porphyrin, a phosphorescent complex of a tetrabenzoporphyrin, a chlorin, a porphyrin-ketone, an aza-porphyrin and a long-decay luminescent complex of iridium(III) or osmium(II).

16. The method of claim 13 wherein interrogations measure photoluminescence lifetime.

17. The method of claim 1 wherein the aliquot within the vessel is pasteurized at 72° C. for 20 seconds and incubated at 30° to 50° C. for up to 24 hours.

18. The method of claim 1 wherein the aliquot within the vessel is pasteurized at 63° C. for 30 minutes and incubated at 30° to 55° C. for up to 24 hours.

19. The method of claim 17 or 18 wherein the aliquot within the vessel is incubated at 30° C.

20. The method of claim 1 wherein pasteurization temperatures are achieved with a dry block heater.

21. The method of claim 1 further comprising the step of adding nutrients effective for promoting growth of at least one thermoduric microorganism to the aliquot prior to pasteurization.

22. A method for comparatively detecting the presence of thermoduric microorganisms and total microorganisms in a product, comprising the steps of:
(a) obtaining a sample of the product,
(b) placing a first aliquot of the sample into a first retention chamber equipped with a first probe sensitive to a thermoduric microorganism metabolite,
(c) placing a second aliquot of the sample into a second retention chamber equipped with a second probe sensitive to a target-analyte,
(d) pasteurizing the first aliquot within the first retention chamber but not the second aliquot,
(e) incubating the pasteurized first aliquot within the first retention chamber and the second aliquot within the second retention chamber for an incubation period, and
(f) periodically interrogating both probes during the incubation period, wherein the interrogations measure changes in the probe reflective of changes in concentration of a thermoduric microorganism metabolite within the first aliquot and changes in concentration of a target-analyte within the second aliquot, with such changes in concentration indicative of the presence of viable thermoduric microorganisms in the first aliquot and the presence of total viable microorganisms in the second aliquot.

23. The method of claim 22 wherein the thermoduric microorganism metabolite and the target-analyte are both oxygen.

24. The method of claim 22 further comprising the steps of (i) converting measured changes in the first probe to a concentration of thermoduric microorganisms in the pasteurized first aliquot prior to incubation based upon a known conversion algorithm, (ii) converting measured changes in the second probe to a concentration of total microorganisms in the second aliquot prior to incubation based upon a known conversion algorithm, and (ii) reporting the ascertained concentration of thermoduric microorganisms and total microorganisms.

25. The method of claim 22 further comprising the step of hermetically sealing the first and second aliquots within their respective retention chambers prior to pasteurization and incubation.

26. The method of claim 22 wherein the product is a food product intended for human consumption.

27. The method of claim 26 wherein the product is milk.

28. The method of claim 22 wherein the first and second retention chambers are defined by separate and independent vials or cuvettes.

29. The method of claim 22 wherein the first and second retention chambers are different wells in a multi-well plate.

30. The method of claim 22 wherein the first and second retention chambers have an elevated surface-to-volume ratio.

31. The method of claim 22 wherein pasteurization is effected at a defined temperature for a defined time period and thermal equilibration of the aliquot in the vessel is achieved within ½ of the pasteurization time period.

32. The method of claim 22 wherein pasteurization is effected at a defined temperature for a defined time period and thermal equilibration of the aliquot in the vessel is achieved within ¼^{th} of the pasteurization time period.

33. The method of claim 29 wherein the multi-well plate has more than 40 wells.

34. The method of claim 22 wherein (i) the first and second retention chambers have open top ends and closed bottom ends, and (ii) each probe is positioned within the respective retention chamber proximate the bottom end.

35. The method of claim 22 wherein the first and second probes are the same type of probe.

36. The method of claim 35 wherein the first and second probes are oxygen sensitive photoluminescent dye.

37. The method of claim 36 wherein the oxygen sensitive photoluminescent dye is an oxygen sensitive transition metal complex.

38. The method of claim 37 wherein the oxygen sensitive transition metal complex is selected from the group consisting of a ruthenium bipyridyl, a ruthenium diphenylphenanotroline, a platinum porphyrin, a palladium porphyrin, a phosphorescent complex of a tetrabenzoporphyrin, a chlorin, a porphyrin-ketone, an aza-porphyrin and a long-decay luminescent complex of iridium(III) or osmium(II).

39. The method of claim 36 wherein interrogations measure photoluminescence lifetime.

40. The method of claim 22 wherein the first aliquot is pasteurized at 72° C. for 20 seconds and both the first and second aliquots are incubated at 30° to 50° C. for up to 24 hours.

41. The method of claim 22 wherein the first aliquot is pasteurized at 63° C. for 30 minutes and both the first and second aliquots are incubated at 30° to 55° C. for up to 24 hours.

42. The method of claim 40 or 41 wherein the first aliquot is incubated at 30° C.

43. The method of claim 22 wherein pasteurization temperatures are achieved with a dry block heater.

44. The method of claim 22 further comprising the step of adding nutrients effective for promoting growth of microorganisms to the first and second aliquots prior to pasteurization and incubation.
